(19) United States
(12) Patent Application Publication
(10) Pub. No.: US 2021/0052831 A1
(43) Pub. Date: Feb. 25, 2021

(54) NEBULIZER

(71) Applicant: DELBio, INC., Taoyuan City (TW)

(72) Inventors: Chun-Hao Hsu, Taoyuan City (TW); Shih-Kai Chien, Taoyuan City (TW); Ke-Cheng Lin, Taoyuan City (TW)

(21) Appl. No.: 15/930,934

(22) Filed: May 13, 2020

(30) Foreign Application Priority Data
Aug. 23, 2019 (CN) 201921384578.6

(51) Int. Cl. A61M 15/00

(52) U.S. Cl. CPC A61M 15/001; A61M 15/0085; A61M 15/0021; A61M 2202/04; A61M 15/009; A61M 2205/3334; A61M 15/0086

(57) ABSTRACT

A nebulizer capable of connecting with a mouthpiece is provided. The nebulizer includes a container, a nebulizing part, a first tube and a second tube. The container is configured to accommodate a liquid. The nebulizing part includes a chamber and a nebulizing element communicating with the container and nebulizing the liquid in the chamber. The first tube connected with the nebulizing part includes a first channel communicating with the chamber and introducing an outside airflow. The second tube is connected with the nebulizing part and the mouthpiece. A second channel of the second tube communicates with the chamber and the mouthpiece. The second tube has an expanding part at a junction of the second channel and the chamber.

### CROSS-REFERENCE TO RELATED APPLICATION

This application claims priority to China Patent Application No. 201921384578.6, filed on Aug. 23, 2019, the entire content of which is incorporated herein by reference for all purposes.

### FIELD OF THE INVENTION

The present disclosure relates to a nebulizer, and more particularly to a nebulizer improving the delivery rate of aerosols.

### BACKGROUND OF THE INVENTION

In the conventional nebulizer, the liquid is nebulized into sprays or aerosols through vibrating or heating, and the aerosol is transferred to the mouthpiece by inhalation. The nebulizer has an air inlet to introduce outside airflow to increase the delivery rate of the aerosol. However, the conventional nebulizer is usually not optimally designed. The configuration of the inlet or channel may easily form an eddy to block the moving path of the aerosols. Then, a part of the aerosols may remain in the channel and cannot be transported to the mouthpiece. In addition, in the conventional nebulizer, the inlet is mostly disposed below the nebulizing area. Consequently, when the remained aerosols are condensed back into the liquid in the nebulizing area so that the air inlet is easily blocked, and thereby reducing the delivery rate of aerosols.

Therefore, there is a need of providing a nebulizer in order to overcome the above drawbacks.

### SUMMARY OF THE INVENTION

An object of the present disclosure provides a nebulizer. The liquid is nebulized in the chamber. Through the design of the angle at the junction of the channel and the chamber, most of the nebulized aerosol is prevented from being blocked and forming an eddy.

An object of the present disclosure provides a nebulizer. In the nebulizer, the perpendicular distance from the air inlet of the channel, which is configured to introduce the outside airflow, to the bottom of chamber is equal to or larger than the perpendicular distance from the nebulizing element to the bottom of chamber. Consequently, the condensed aerosol is prevented from flowing to the channel and obstructing the intake.

Another object of the present disclosure provides a nebulizer. In the channel of the nebulizer, the aperture of the air inlet is larger than the aperture of the air outlet. Therefore, the aperture the channel is gradually narrowed in the moving path of fluid, which improves the velocity of the fluid moving in the channel.

In accordance with an aspect of the present disclosure, there is provided a nebulizer capable of connecting with a mouthpiece. The nebulizer includes a container, a nebulizing part, a first tube and a second tube. The container is configured to accommodate a liquid. The nebulizing part includes a nebulizing element and a chamber, and the nebulizing element communicates with the container and nebulizes the liquid in the chamber. The first tube is connected with the nebulizing part, the first tube includes a first channel communicating with the chamber, and the first channel is configured to introduce an outside airflow. The second tube is connected with the nebulizing part and the mouthpiece. A second channel of the second tube communicates with the chamber and the mouthpiece. The second tube has an expanding part at a junction of the second channel and the chamber.

In accordance with another aspect of the present disclosure, there is further provided a nebulizer. The nebulizer includes a container, a nebulizing part, a first tube and a second tube. The container is configured to accommodate a liquid. The nebulizing part includes a nebulizing element and a chamber, and the nebulizing element communicates with the container and nebulizes the liquid in the chamber. The first tube is connected with the nebulizing part, and the first tube is configured to introduce an outside airflow to the chamber. The second tube is connected with the nebulizing part. The second tube communicates with the chamber through an inlet, and there is an acute angle equal to or greater than 60 degrees between an end face of the inlet and a sidewall of the second tube.

In accordance with another aspect of the present disclosure, there is further provided a nebulizer capable of connecting with a mouthpiece. The nebulizer includes a container, a nebulizing part, a first tube and a second tube. The container is configured to accommodate a liquid. The nebulizing part includes a nebulizing element and a chamber, and the nebulizing element communicates with the container. The first tube is connected with the nebulizing part. The first tube includes a first channel communicating with the chamber, and the first channel has an outlet adjacent to the chamber. The outlet is higher than a bottom of the chamber. The second tube is connected with the nebulizing part and the mouthpiece. The chamber communicates with the mouthpiece through a second channel of the second tube. The second channel is gradually narrowed along a direction from the chamber to the mouthpiece.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 is a schematic cross-sectional view illustrating a nebulizer according to a first embodiment of the present disclosure;

FIGS. 2A, 2B, 4 and 5A are schematic cross-sectional structural views illustrating variants of the nebulizer of FIG. 1;

FIG. 3 is a schematic cross-sectional view illustrating a nebulizer according to a second embodiment of the present disclosure; and

FIG. 5B is a schematic cross-sectional view illustrating a variant of the nebulizer of FIG. 3.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

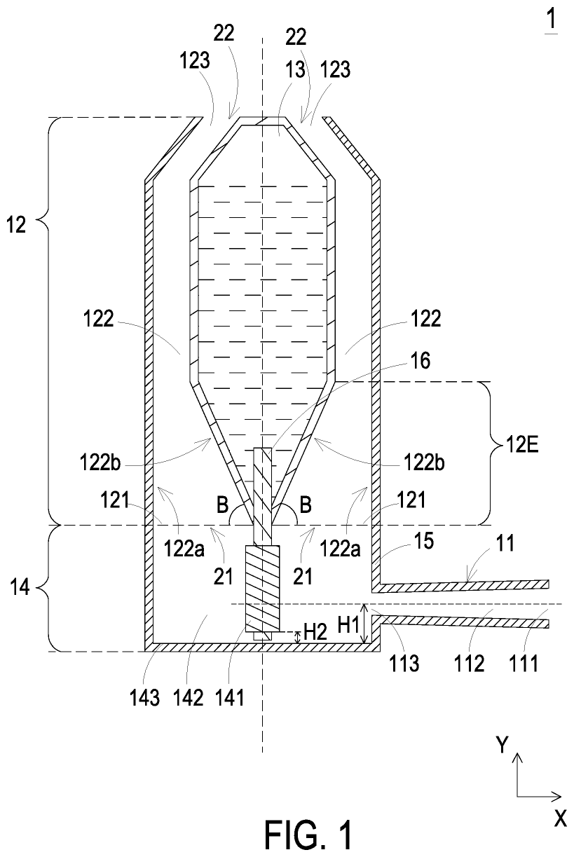

Please refer to FIG. 1. FIG. 1 is a schematic cross-sectional view illustrating a nebulizer according to a first embodiment of the present disclosure. As shown in FIG. 1, the nebulizer 1 can be connected with a mouthpiece (not shown), and the nebulizer 1 includes a first tube 11, a second tube 12, a liquid supply container 13 and a nebulizing part 14. The liquid supply container 13 is configured to accommodate the liquid. The nebulizing part 14 includes a nebulizing element 141 and a chamber 142. The nebulizing element 141 communicates with the liquid supply container 13, and the nebulizing element 141 nebulizes the liquid in the chamber 142.

The first tube 11 is connected with the nebulizing part 14, and the first tube 11 has a first channel 112. The first channel 112 is configured to introduce the outside airflow and communicates with the chamber 142. The first channel 112 has a first air inlet 111 and a first air outlet 113. The first channel 112 introduces the outside airflow through the first air inlet 111 and guides the outside airflow to the chamber 142 through the first air outlet 113.

The second tube 12 is connected with the nebulizing part 14 and the mouthpiece, and the second tube 12 has a second channel 122 configured to allow the chamber 142 and the mouthpiece to communicates with each other. The second channel 122 has a second air inlet 121 and a second air outlet 123. The second channel 122 communicates with the chamber 142 through the second air inlet 121 and communicates with the mouthpiece through the second air outlet 123. The second tube 12 has an expanding part 12E at the junction of the second channel 122 and the chamber 142. The bottom surface 143 of the nebulizing part 14 is a plane surface, and the normal direction of the bottom surface 143 is the first direction C. The first direction C is perpendicular to the second direction D. There is an acute angle between a sidewall 122b of the expanding part 12E of the second tube 12 and the second direction D. Moreover, the first direction C is parallel to the y axis, and the second direction D is parallel to the x axis. In an embodiment of the present disclosure, there is a first end face 21 at the junction of the second channel 122 and the chamber 142. There is an acute angle between the first end face 21 and the sidewall 122b of the expanding part 12E, where the normal direction of the first end face 21 is the first direction C.

When the nebulizer 1 needs to release the nebulized aerosols, the liquid supply container 13 supplies liquid to the chamber 142, and the nebulizing element 141 nebulizes the liquid into aerosols. Since the first air outlet 113 of the first tube 11 is disposed corresponding to the nebulizing part 141, and the outside airflow introduced through the first air inlet 111, the first channel 112 and the first air outlet 113 can directly flow toward the nebulizing element 141. Further, the introduced outside airflow drives the nebulized aerosols to move through the second channel 122 and release from the second air outlet 123, where the outside airflow is for example but not limited to mix with the aerosols and form a mixture gas. As mentioned above, the second tube 12 has the structure of expanding part, and there is an acute angle between the sidewall of the expanding part and the second direction D. Therefore, most of the aerosols are prevented from forming an eddy at the second air inlet 121, which allows the aerosols to largely and rapidly flow from the chamber 142 to the second air outlet 123.

In an embodiment, the first tube 11 may be perpendicular to the second tube 12. In an embodiment, the first tube 11 is disposed on a side of the nebulizing part 14. In other words, on the y axis, the position of the first air outlet 113 of the first tube 11 is higher than that of the bottom surface 143 of the nebulizing part 14. Through the improvement achieved by above embodiments, even if the aerosols are slightly condensed, the aerosols can be nebulized again when flowing back to the chamber 142. Even if a part of the aerosols flow back to the bottom of the chamber 142, this part of the aerosols will not easily flow into the first channel 112 and obstruct the intake.

As shown in Table 1. In the conventional nebulizer, when the nebulizer stops releasing aerosols, more liquid remains in the first channel (i.e., the channel for introducing the outside airflow), the second channel (i.e., the channel for releasing the aerosols) and the chamber due to the phenomenon of the reflux of the aerosols and the condensed liquid flowing into the first channel. On the contrary, in the nebulizer 1 of the present disclosure, there is no liquid remained in the first channel 112, and only little liquid remains in the second channel 122 and the chamber 142. Therefore, the amount of the released medicine is increased.

TABLE 1

| | the amount of residual liquid (mg) | |
|---|---|---|
| | | in the second channel |
| | in the first channel | and the chamber |
| the conventional | 64.3 | 32.3 |
| nebulizer | 65.2 | 47.8 |
| | 42.7 | 37.6 |
| the nebulizer 1 of the | 0 | 3.2 |
| present disclosure | 0 | 5.1 |
| | 0 | 2.6 |

In an embodiment, the aperture of the first air inlet 111 is larger than the aperture of the first air outlet 113. Preferably, the aperture of the first channel 112 is gradually narrowed in the moving path of the airflow, which improves the velocity of the airflow moving in the first channel 112. In an embodiment, the aperture of the second air inlet 121 is larger than the aperture of the second air outlet 123. Preferably, the second channel 122 is gradually narrowed in width along the direction from the nebulizing part 14 to the mouthpiece. Accordingly, the pipe diameter of the second channel 122 is gradually narrowed in the moving path of the sprays or aerosols, which improves the velocity of the sprays or aerosols moving in the second channel 122. In an embodiment, there is a first end face 21 at the junction of the second channel 122 and the chamber 142, and there is a second end face 22 at the junction of the second channel 122 and the mouthpiece. The area of the first end face 21 is larger than that of the second end face 22.

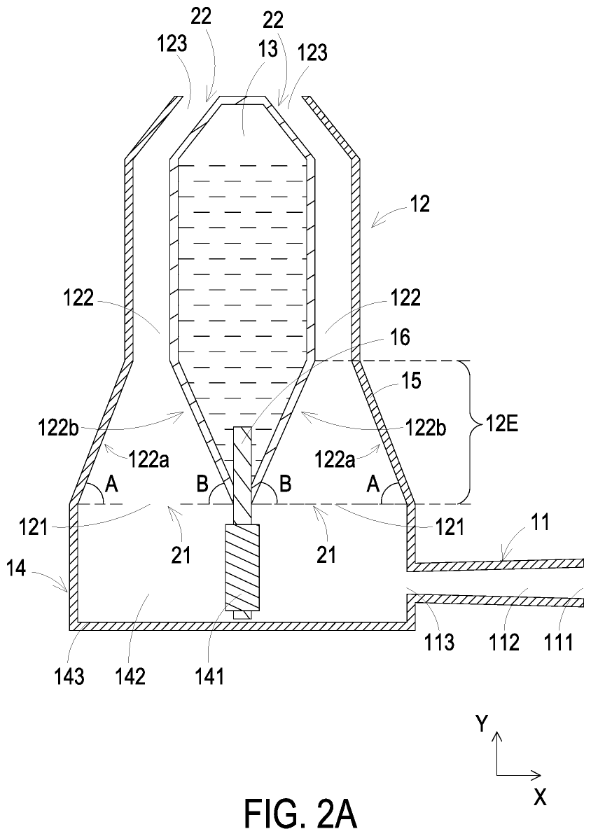

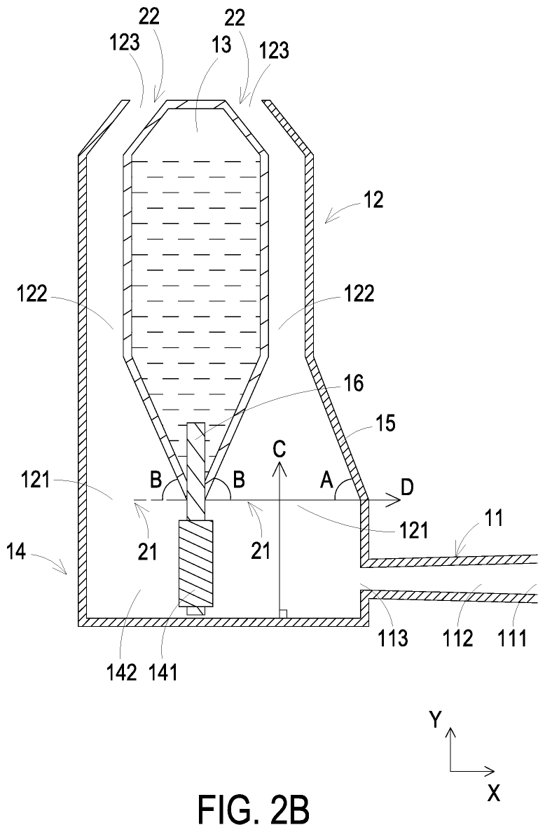

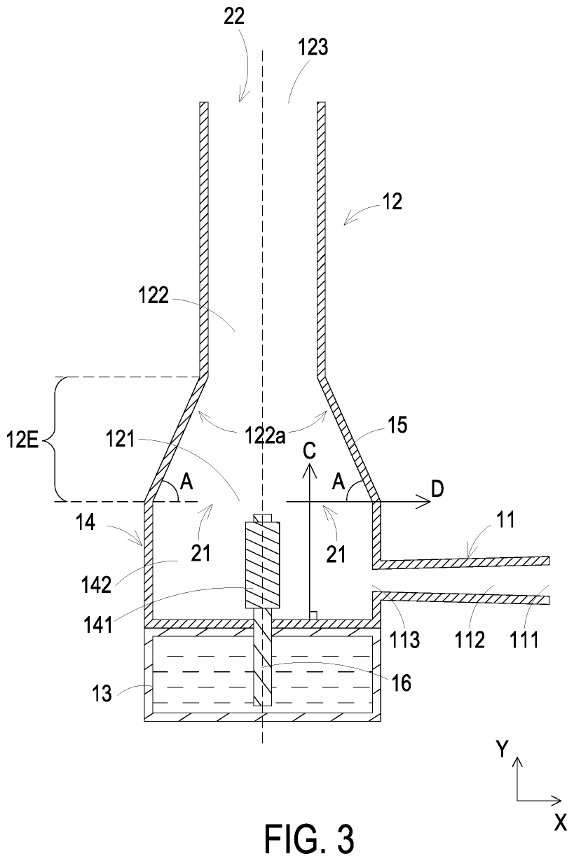

Please refer to FIGS. 1, 2A, 2B and 3. FIGS. 2A and 2B are schematic cross-sectional views illustrating variants the nebulizer of FIG. 1, and the main difference is the aspect of the expanding part 12E of the second tube 12. In an embodiment, as shown in FIG. 3, the second tube 12 has a sidewall 122a at the expanding part 12E at the junction of the second channel 122 and the chamber 142. The sidewall 122a is a part of the housing 15 of the nebulizer 1 or is neighboring to the housing 15 of the nebulizer 1, and the sidewall 122a is far away from the center line of the nebulizer 1. The center line of the nebulizer 1 is parallel to the first direction C. There is an acute angle A between the sidewall 122a of the second tube 12 and the second direction D, and the sidewall 122a is not perpendicular to the first direction C. In an embodiment of the present disclosure, there is a first end face 21 at the junction of the second channel 122 and the chamber 142, and there is an acute angle A between the first end face 21 and the sidewall 122a of the expanding part 12E. The normal direction of the first end face 21 is the first direction C.

In an embodiment, as shown in FIG. 1, the second channel 122 of the second tube 12 can accommodate the liquid supply container 13. The second tube 12 has a sidewall 122b opposite to the sidewall 122a at the junction of the second channel 122 and the chamber 142, and the sidewall 122b not perpendicular to the first direction C. The sidewall 122b is neighboring to the center line of the nebulizer 1 and forms the bottom of the liquid supply container 13. In the embodiment of FIG. 1, the acute angle between the sidewall 122b of the second tube 12 and the second direction D includes an acute angle B. In other words, there is a first end face 21 at the junction of the second channel 122 and the chamber 142, and there is an acute angle B between the first end face 21 and the sidewall 122b of the expanding part 12E. The normal direction of the first end face 21 is the first direction C. In another embodiment, as shown in FIG. 2A, the acute angle between the sidewall of the second tube 12 and the second direction D includes the acute angle A and the acute angle B. The expanding part 12E of the second tube 12 is in the shape of a horn. Namely, the acute angle between the sidewall of the expanding part 12E of the second tube 12 and the first end face 21 includes the acute angle A and the acute angle B. In another embodiment, as shown in FIG. 2B, the sidewall at different parts of the second channel 122 of the second tube 12 is different. Accordingly, the aspect of the acute angle between each sidewall and the second direction D is different as well. For example but not exclusively, there are acute angles A and B between the sidewall and the second direction D in a part of the second channel 122, and there is only acute angle B between the sidewall and the second direction D in another part of the second channel 122. In an embodiment, the acute angles A and B are equal to or larger than 60 degrees. In an embodiment, the sidewall of the second tube is adjacent to the chamber.

In addition, the liquid supply container 13 is disposed inside or outside the second tube 12 according to actual requirements. In an embodiment, as shown in FIGS. 3, 4 and 5B, the liquid supply container 13 is disposed outside the second tube 12 and communicates with the nebulizing part 14.

In an embodiment, the nebulizer 1 further includes a liquid guiding element 16. One end of the liquid guiding element 16 is disposed inside the liquid supply container 13 and is contacted with the liquid contained in the liquid supply container 13, and the other end of the liquid guiding element 16 is disposed within the chamber 142. The nebulizing element 141 is sleeved on the liquid guiding element 16. Consequently, the end of the liquid guiding element 16 inside the liquid supply container 13 draws the liquid, and the drawn liquid is transmitted to the other end of the liquid guiding element 16 within the chamber 142, which allows the nebulizing part 141 to nebulize the liquid into the aerosols. The liquid guiding element 16 is for example but not limited to a wick held by the opening of the liquid supply container 13, and the wick is configured to transfer the liquid to the nebulizing element covering the wick.

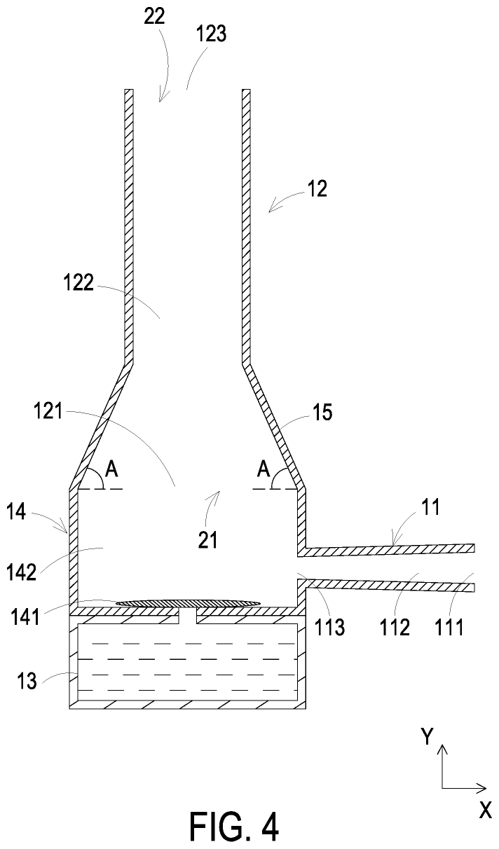

In an embodiment of the present invention, in order to allow the outside airflow introduced from the first air outlet 113 to directly flow toward the nebulizing element 141 and to drive the nebulized aerosols, the first air outlet 113 is disposed corresponding to the nebulizing element 141. For example but not exclusively, the central extension line of the first channel 112 intersects with the nebulizing element 141. In an embodiment, the perpendicular distance of the first air outlet 113 of the first tube 11 to the bottom of the chamber 142 is the first height H1, and the perpendicular distance of the nebulizing element 141 to the bottom of the chamber 142 is the second height H2. The first height H1 is larger than or equal to the second height H2. Therefore, the outside airflow introduced from the first air outlet 113 is facilitated to drive the nebulized aerosols, and little amount of condensed liquid flowing back to the chamber 142 is prevented from flowing into the first channel 112. In above embodiments, the nebulizing element 141 is a heater, which nebulizes the liquid through heating. In an embodiment, the projection of the periphery of the first air outlet 113 on the liquid guiding element 16 is located between the center point of the heater and the end of the heater farthest from the second tube 12. In an embodiment, as shown in FIG. 4, the nebulizing element 141 is a vibrating piece, which nebulizes the liquid through vibrating the mesh plate or ultrasonic. One side of the vibrating piece communicates with the liquid supply container 13, and the other side of the vibrating piece communicates with the chamber 142. In addition, the liquid in the liquid supply container 13 may be transported to the chamber 142 through a transporting device (not shown) for allowing the vibrating piece to nebulize the liquid, but not limited thereto. The specific embodiments of the nebulizing element 141 listed above is used to illustrate the relative position between the nebulizing element 141 and the first air outlet 113. However, the possible embodiment of the nebulizing element 141 is not limited thereto.

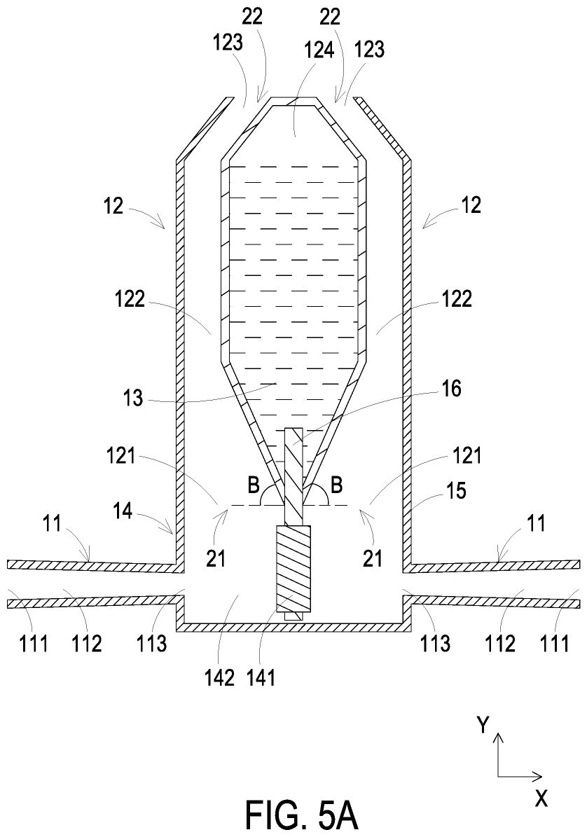

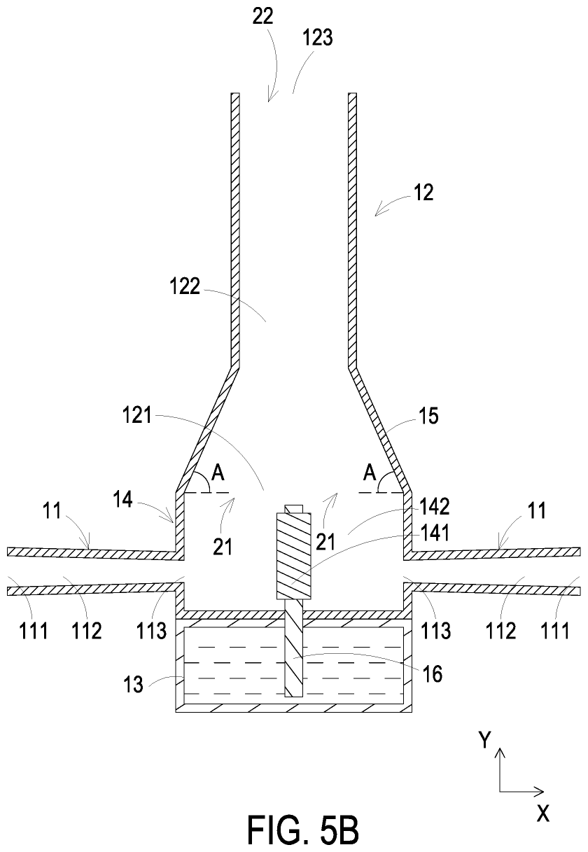

Moreover, the number of the first tube 11 of the nebulizer 1 is not limited to one. The nebulizer 1 may include a plurality of first tubes 11 and a plurality of second tubes 12. The plurality of first tubes 11 is disposed opposite, and each first tube 11 communicates with the corresponding second tube 12 through the chamber 142. The plurality of second tubes 12 may communicate with each other or not. In an embodiment, a separator 124 is disposed among the plurality of the second tubes 12 for separating the plurality of the second tubes 12 from each other. For example, in the variant shown in FIG. 5A, the nebulizer 1 includes two first tubes 11 and two second tubes 12. The separator 124 is disposed between the two second tubes 12, and thus the two second tubes 12 do not communicate with each other. The two first tubes 11 are respectively connected with the corresponding second tube 12 through the chamber 142. In addition, the separator 124 may be the liquid supply container 13. In the variant shown in FIG. 5B, the nebulizer 1 includes two first tubes 11 and one second tubes 12, and two first tubes 11 both communicate with the second tube 12 through the chamber 142.

From the above descriptions, the present invention provides a nebulizer. The liquid is nebulized in the chamber. Through the design of the angle at the junction of the channel and the chamber, most of the nebulized aerosol is prevented from being blocked and forming an eddy. Moreover, the perpendicular distance from the air inlet of the channel, which is configured to introduce the outside airflow, to the bottom of chamber is equal to or larger than the perpendicular distance from the nebulizing element to the bottom of chamber. Consequently, the condensed aerosol is prevented from flowing to the channel and obstructing the intake. Furthermore, in the channel of the nebulizer, the aperture of the air inlet is larger than the aperture of the air outlet. Therefore, the aperture the channel is gradually narrowed in the moving path of fluid, which improves the velocity of the fluid moving in the channel.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A nebulizer capable of connecting with a mouthpiece, comprising:
a container configured to accommodate a liquid;
a nebulizing part comprising a nebulizing element and a chamber, wherein the nebulizing element communicates with the container and nebulizes the liquid in the chamber;
a first tube connected with the nebulizing part, wherein the first tube comprises a first channel communicating with the chamber, and the first channel is configured to introduce an outside airflow; and
a second tube connected with the nebulizing part and the mouthpiece, wherein a second channel of the second tube communicates with the chamber and the mouthpiece, and the second tube has an expanding part at a junction of the second channel and the chamber.

2. The nebulizer according to claim 1, wherein a sidewall of the expanding part is not perpendicular to a normal direction of a bottom surface of the nebulizing part.

3. The nebulizer according to claim 2, wherein the expanding part has another sidewall opposite to the sidewall, and the another sidewall is not perpendicular to the normal direction.

4. The nebulizer according to claim 2, wherein there is an acute angle between the sidewall and a first end face at the junction of the second channel and the chamber.

5. The nebulizer according to claim 4, wherein the acute angle is equal to or larger than 60 degrees.

6. The nebulizer according to claim 1, wherein there is a first end face at the junction of the second channel and the chamber, there is a second end face at the junction of the second channel and the mouthpiece, and an area of the first end face is larger than an area of the second end face.

7. The nebulizer according to claim 1, wherein the first channel has a first outlet communicating with the chamber, and a perpendicular distance from the first outlet to a bottom of the chamber is equal to or larger than a perpendicular distance from the nebulizing element to the bottom of the chamber.

8. The nebulizer according to claim 7, wherein the first channel has a first inlet configured to introduce the outside airflow, and the first channel is gradually narrowed from the first inlet to the first outlet.

9. The nebulizer according to claim 1, further comprising a plurality of the first tubes, wherein the plurality of the first tubes are disposed opposite.

10. The nebulizer according to claim 9, further comprising a plurality of the second tubes, wherein the plurality of the second tubes do not communicate with each other, and each of the first tubes communicates with the corresponding second tube through the chamber.

11. The nebulizer according to claim 1, wherein a bottom of the container is formed by the sidewall of the expanding part.

12. The nebulizer according to claim 1, wherein a wick is held by an opening of the container, and the wick is configured to transfer the liquid to the nebulizing element covering the wick.

13. The nebulizer according to claim 1, wherein the nebulizing element is a vibrating piece, and the container communicates with the chamber through the vibration piece.

14. The nebulizer according to claim 1, wherein the second channel is gradually narrowed along a direction from the chamber to the mouthpiece.

15. A nebulizer, comprising:
a container configured to accommodate a liquid;
a nebulizing part comprising a nebulizing element and a chamber, wherein the nebulizing element communicates with the container and nebulizes the liquid in the chamber;
a first tube connected with the nebulizing part, wherein the first tube is configured to introduce an outside airflow to the chamber; and
a second tube connected with the nebulizing part, wherein the second tube communicates with the chamber through an inlet, and there is an acute angle equal to or greater than 60 degrees between an end face of the inlet and a sidewall of the second tube.

16. The nebulizer according to claim 15, wherein the sidewall of the second tube is adjacent to the chamber.

17. A nebulizer capable of connecting with a mouthpiece, comprising:
a container configured to accommodate a liquid;
a nebulizing part comprising a nebulizing element and a chamber, wherein the nebulizing element communicates with the container;
a first tube connected with the nebulizing part, wherein the first tube comprises a first channel communicating with the chamber, the first channel has an outlet adjacent to the chamber, and the outlet is higher than a bottom of the chamber; and
a second tube connected with the nebulizing part and the mouthpiece, wherein the chamber communicates with the mouthpiece through a second channel of the second tube, and the second channel is gradually narrowed along a direction from the chamber to the mouthpiece.

18. The nebulizer according to claim 17, wherein a normal direction of a bottom surface of the nebulizing part is perpendicular to a direction, and there is an acute angle between a sidewall of the second tube and the direction.
